(19) United States
(12) Patent Application Publication
(10) Pub. No.: US 2025/0345239 A1
(43) Pub. Date: Nov. 13, 2025

(54) METHOD AND APPARATUS FOR DETERMINING ABDOMINAL ACUPUNCTURE POINTS, MOXIBUSTION ROBOT, AND STORAGE MEDIUM

(71) Applicant: Jianghan University, Wuhan City (CN)

(72) Inventors: Qiang HE, Wuhan City (CN); Zhihao FENG, Wuhan City (CN); Hongxing ZHANG, Wuhan City (CN); Qian TU, Wuhan City (CN)

(21) Appl. No.: 19/097,187

(22) Filed: Apr. 1, 2025

(30) Foreign Application Priority Data
May 10, 2024 (CN) 202410571306.6

(51) Int. Cl. A61H 39/02; A61H 39/06; G16H 40/63

(52) U.S. Cl. CPC A61H 39/02; A61H 39/06; G16H 40/63; A61H 2201/5007

(57) ABSTRACT

A method and apparatus for determining abdominal acupuncture points, a moxibustion robot, and a storage medium are provided, which belong to the technical field of identification of acupuncture points. The method includes: acquiring human body key feature points of a human body image; determining a plurality of meridians on an abdomen based on the human body key feature points; and determining abdominal acupuncture points based on a topological relationship of the meridians and acupuncture points. Abdominal acupuncture points are determined by means of the meridians such that the technical problem of inaccurate locating of acupuncture points on the abdomen without texture feature due to the use of a deep learning model is avoided, and the locating accuracy of the abdominal acupuncture points is improved.

### CROSS REFERENCE TO RELATED APPLICATION

This patent application claims the benefit and priority of Chinese Patent Application No. 202410571306.6, filed with the China National Intellectual Property Administration on May 10, 2024, the disclosure of which is incorporated by reference herein in its entirety as part of the present application.

### TECHNICAL FIELD

The present disclosure relates to the technical field of identification of acupuncture points, and in particular, to a method and apparatus for determining abdominal acupuncture points, a moxibustion robot, and a storage medium.

### BACKGROUND

As people are paying increasing attention to the physical health, an increasing number of people select moxibustion as a treatment therapy for physical complaints. Moxibustion, as a Chinese traditional medical treatment therapy, has been passed down to now, and a plurality of acupuncture and moxibustion techniques have been derived. Among them, abdominal acupuncture therapy is a process of applying acupuncture to corresponding acupuncture points on the abdomen of a human to treat various diseases on the abdomen.

An existing mainstream method for locating abdominal acupuncture points is to implement detection by training a deep learning model. However, this method, for the abdomen having few texture features, cannot well learn features, leading to inaccurate locating of abdominal acupuncture points and then affecting the moxibustion treatment. In order to implement accurate locating on the abdomen, manual acupuncture point location is the most common way. That is, abdominal acupuncture points are determined and moxibustion is applied by human experience. However, manual acupuncture point location is inefficient and relies on the human experience. When the experience is insufficient, the locating of the abdominal acupuncture points is inaccurate.

Therefore, there is an urgent need to provide a method and apparatus for determining abdominal acupuncture points, a moxibustion robot, and a storage medium to improve the accuracy of the determined abdominal acupuncture points.

### SUMMARY

In view of the above, it is necessary to provide a method and apparatus for determining abdominal acupuncture points, a moxibustion robot, and a storage medium to address the technical problem of low accuracy the determined abdominal acupuncture points in the prior art.

In an aspect, the present disclosure provides a method for determining abdominal acupuncture points, including:
acquiring human body key feature points of a human body image;
determining a plurality of meridians on an abdomen based on the human body key feature points; and
determining abdominal acupuncture points based on a topological relationship of the meridians and acupuncture points.

In some possible implementations, the human body key feature points include a first-type human body key feature point and a second-type human body key feature point; and the acquiring human body key feature points of a human body image includes:
extracting a key point from the human body image based on a Mediapipe extraction model to determine the first-type human body key feature point; and
training an initial detection model to obtain a target detection model, and extracting a key point from the human body image based on the target detection model to determine the second-type human body key feature point.

In some possible implementations, the first-type human body key feature point includes a nose; the second-type human body key feature point includes a left nipple, a right nipple, and a navel; and the meridians include a Conception Vessel, a left kidney meridian of foot-shaoyin, a right kidney meridian of foot-shaoyin, a left stomach meridian of foot-yangming, a right stomach meridian of foot-yangming, a left spleen meridian of foot-taiyin, and a right spleen meridian of foot-taiyin.

In some possible implementations, the determining a plurality of meridians on an abdomen based on the human body key feature points includes:
determining a first connecting line of the nose and the navel, and using the first connecting line as the Conception Vessel;
drawing a first parallel line and a second parallel line parallel to the first connecting line through the left nipple and the right nipple, respectively, and using the first parallel line as the left spleen meridian of foot-taiyin and the second parallel line as the right spleen meridian of foot-taiyin;
determining a third parallel line parallel to the first connecting line and having an equal distance to the first connecting line and the first parallel line, and using the third parallel line as the left stomach meridian of foot-yangming;
determining a fourth parallel line parallel to the first connecting line and having an equal distance to the first connecting line and the second parallel line, and using the fourth parallel line as the right stomach meridian of foot-yangming; and
determining a fifth parallel line and a sixth parallel line having a distance to the first connecting line as a preset distance, where the fifth parallel line is located between the first connecting line and the third parallel line, and the sixth parallel line is located between the first connecting line and the fourth parallel line; and using the fifth parallel line as the left kidney meridian of foot-shaoyin and the sixth parallel line as the right kidney meridian of foot-shaoyin.

In some possible implementations, the topological relationship includes positional relationships between the meridians and the acupuncture points, a horizontal topological relationship between the meridians, and a vertical topological relationship between the acupuncture points; and the determining the abdominal acupuncture points based on a topological relationship of the meridians and acupuncture points includes:
acquiring a first actual pixel distance and a first Chinese-system distance between the left nipple and the right nipple, and determining a horizontal cun based on the first actual pixel distance and the first Chinese-system distance;
acquiring a second actual pixel distance and a second Chinese-system distance between a center point of the navel and a midpoint of the left nipple and the right nipple, and determining a direct cun based on the second actual pixel distance and the second Chinese-system distance; acquiring the positional relationships;
determining the horizontal topological relationship based on the horizontal cun, and determining the vertical topological relationship based on the direct cun; and
determining the abdominal acupuncture points based on the positional relationships, the horizontal topological relationship, and the vertical topological relationship.

In some possible implementations, the abdominal acupuncture points include Shenque, Xiawan, Zhongwan, Shangwan, Qihai, Guanyuan, Zhongji, left Chengman, left Liangmen, left Taiyi, left Tianshu, left Shuidao, left Guilai, right Chengman, right Liangmen, right Taiyi, right Tianshu, right Shuidao, right Guilai, left Daheng, left Fujie, right Daheng, right Fujie, left Futonggu, left Yindu, left Shangqu, left huangshu, left Qixue, left Dahe, right Futonggu, right Yindu, right Shangqu, right huangshu, right Qixue, and right Dahe;
the positional relationships are as follows: Shenque, Xiawan, Zhongwan, Shangwan, Qihai, Guanyuan, and Zhongji are located on the Conception Vessel; left Chengman, left Liangmen, left Taiyi, left Tianshu, left Shuidao, and left Guilai are located on the left stomach meridian of foot-yangming; right Chengman, right Liangmen, right Taiyi, right Tianshu, right Shuidao, and right Guilai are located on the right stomach meridian of foot-yangming; left Daheng and left Fujie are located on the left spleen meridian of foot-taiyin; right Daheng and right Fujie are located on the right spleen meridian of foot-taiyin; left Futonggu, left Yindu, left Shangqu, left huangshu, left Qixue, and left Dahe are located on the left kidney meridian of foot-shaoyin; and right Futonggu, right Yindu, right Shangqu, right huangshu, right Qixue, and right Dahe are located on the right kidney meridian of foot-shaoyin;
the horizontal topological relationship is as follows: distances of the Conception Vessel to the left stomach meridian of foot-yangming and the right stomach meridian of foot-yangming are 2 horizontal cun; distances of the Conception Vessel to the left spleen meridian of foot-taiyin and the right spleen meridian of foot-taiyin are 4 horizontal cun; and distances of the Conception Vessel to the left kidney meridian of foot-shaoyin and the right kidney meridian of foot-shaoyin are 0.5 horizontal cun; and
the vertical topological relationship is as follows: Xiawan, Zhongwan, and Shangwan are located between Shenque and the nose; a distance between Xiawan and Shenque is 2 direct cun; a distance between Zhongwan and Shenque is 4 direct cun; a distance between Shangwan and Shenque is 5 direct cun; Qihai, Guanyuan, and Zhongji are located between Shenque and a foot; a distance between Qihai and Shenque is 1.5 direct cun; a distance between Guanyuan and Shenque is 3 direct cun; a distance between Zhongji and Shenque is 4 direct cun; left Chengman, right Chengman, and Shangwan have a same vertical coordinate; left Liangmen, right Liangmen, left Yindu, right Yindu, and Zhongwan have a same vertical coordinate; left Taiyi, right Taiyi, left Shangqu, right Shangqu, and Xiawan have a same vertical coordinate; left Tianshu, right Tianshu, left Daheng, right Daheng, left Huangshu, right Huangshu, and Shenque have a same vertical coordinate; left Fujie, right Fujie, and Qihai have a same vertical coordinate; left Shuidao, right Shuidao, left Qixue, right Qixue, and Guanyuan have a same vertical coordinate; and left Guilai, right Guilai, left Dahe, right Dahe, and Zhongji have a same vertical coordinate.

In some possible implementations, the first-type human body key feature point further includes a left shoulder and a right shoulder; and before the determining abdominal acupuncture points based on a topological relationship of the meridians and acupuncture points, the method further includes:
acquiring a connecting line between the left shoulder and the right shoulder, and determining a midpoint of the connecting line;
drawing a straight line perpendicular to the connecting line through the connection midpoint; and
determining whether an angle deviation value between the straight line and the Conception Vessel is less than a deviation threshold, and if yes, indicating that the Conception Vessel is accurate.

In another aspect, the present disclosure further provides an apparatus for determining abdominal acupuncture points, including:
a key feature point acquisition unit configured to acquire human body key feature points of a human body image;
a meridian determination unit configured to determine a plurality of meridians on an abdomen based on the human body key feature points; and
an abdominal acupuncture point determination unit configured to determine abdominal acupuncture points based on a topological relationship of the meridians and acupuncture points.

In another aspect, the present disclosure further provides a moxibustion robot, including an acupuncture point location subsystem for determining abdominal acupuncture points and a moxibustion implementation subsystem for applying moxibustion based on the abdominal acupuncture points, where the acupuncture point location subsystem includes a memory and a processor;
the memory is configured to store a program; and
the processor is coupled with the memory and configured to execute the program stored in the memory to implement the steps of the method for determining abdominal acupuncture points in any of the above possible implementations.

In another aspect, the present disclosure further provides a computer readable storage medium, storing a program or instructions which, when executed by a processor, cause(s) the steps of the method for determining abdominal acupuncture points in any of the above possible implementations to be implemented.

The above embodiments have the following beneficial effects: the method for determining abdominal acupuncture points provided in the present disclosure can determine a plurality of meridians on an abdomen based on human body key feature points and then determine abdominal acupuncture points based on a topological relationship of the meridians and acupuncture points. In this way, it is realized that the abdominal acupuncture points are determined by means of the meridians such that the technical problem of inaccurate locating of acupuncture points on the abdomen without texture feature due to the use of a deep learning model is avoided, and the locating accuracy of the abdominal acupuncture points is improved.

Further, the present disclosure omits a marking process without marking acupuncture points. Therefore, the problem of inaccurate locating caused by inaccurate marking can be avoided. The locating accuracy of the abdominal acupuncture points is further improved, and the locating efficiency of the abdominal acupuncture points is improved.

### BRIEF DESCRIPTION OF THE DRAWINGS

In order to describe the technical solutions in the embodiments of the present disclosure more clearly, the accompanying drawings required to describe the embodiments are briefly described below. Apparently, the accompanying drawings in the following description show merely some embodiments of the present disclosure, and those skilled in the art may still derive other accompanying drawings from these accompanying drawings without creative efforts.

FIG. 1 is a flowchart of one embodiment of a method for determining abdominal acupuncture points provided by the present disclosure;

FIG. 2 is a flowchart of one embodiment of S101 in FIG. 1 of the present disclosure;

FIG. 3 is a flowchart of one embodiment of S102 in FIG. 1 of the present disclosure;

FIG. 4 is a structural schematic diagram of one embodiment of meridians provided by the present disclosure;

FIG. 5 is a flowchart of one embodiment of S103 in FIG. 1 of the present disclosure;

FIG. 6 is a flowchart of one embodiment of validating the accuracy of the Conception Vessel provided by the present disclosure;

FIG. 7 is a structural schematic diagram of one embodiment of an apparatus for determining abdominal acupuncture points provided by the present disclosure; and

FIG. 8 is a structural schematic diagram of one embodiment of a moxibustion robot provided by the present disclosure.

### DETAILED DESCRIPTION OF THE EMBODIMENTS

The technical solution of the present disclosure will be described below clearly and completely with reference to the drawing in the embodiment of the present disclosure. Apparently, the described embodiments are merely a part rather than all of the embodiments of the present disclosure. All other embodiments obtained by those skilled in the art based on the embodiments of the present disclosure without creative efforts shall fall within the protection scope of the present disclosure.

It should be understood that the schematic accompanying drawings are not drawn based on a scale of a real object. The flowcharts used in the present disclosure show the operations implemented according to some embodiments of the present disclosure. It should be understood that the operations in the flowcharts may be performed out of sequence, and the steps without a logical context relationship may be performed in a reverse sequence or at the same time. Moreover, those skilled in the art may add one or more other operations to the flowcharts or remove one or more operations from the flowcharts based on the content of the present disclosure. Some of the block diagrams shown in the accompanying drawings are functional entities, and do not necessarily correspond to physically or logically independent entities. These functional entities may be implemented in the form of software, or implemented in one or more hardware modules or integrated circuits, or implemented in different networks and/or processor systems and/or microcontroller systems.

The terms such as “first”, “second”, and the like described in the embodiments of the present disclosure are used only for the purpose of description and cannot be construed as indicating or implying relative importance, or implicitly indicating the number of the indicated technical features. Therefore, technical features defined by “first” and “second” may explicitly or implicitly include at least one of the features.

When an “embodiment” is mentioned herein, specific features, structures, or characteristics described in conjunction with the embodiment may be included in at least one embodiment of the present disclosure. The term appearing in different parts of the specification does not necessarily refer to the same embodiment or an independent or alternative embodiment exclusive of other embodiments. It may be explicitly or implicitly appreciated by those skilled in the art that the embodiments described herein may be combined with other embodiments.

The present disclosure provides a method and apparatus for determining abdominal acupuncture points, a moxibustion robot, and a storage medium, which will be separately described below.

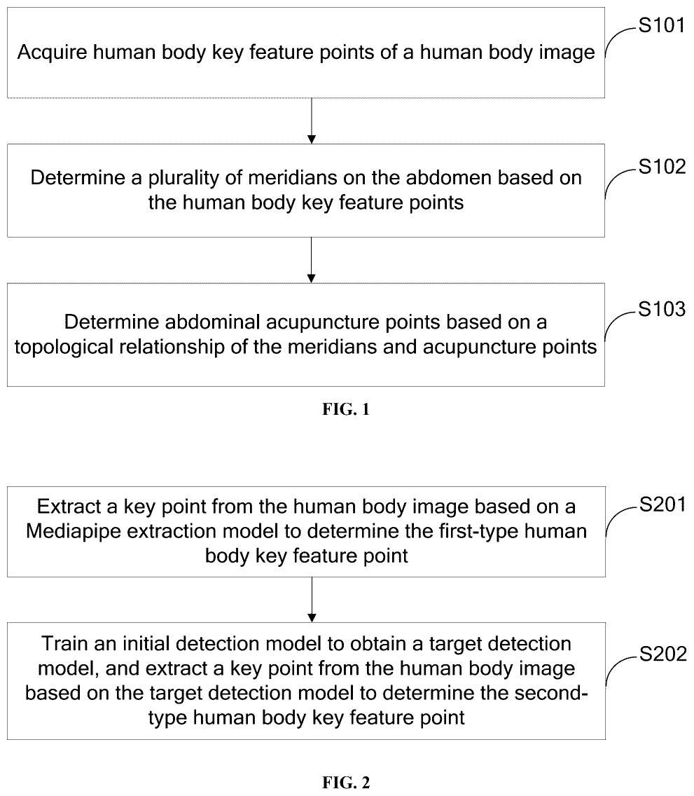

FIG. 1 is a flowchart of one embodiment of a method for determining abdominal acupuncture points provided by an embodiment of the present disclosure. As shown in FIG. 1, the method for determining abdominal acupuncture points includes the following steps.

In step S101, human body key feature points of a human body image are acquired.

In step S102, a plurality of meridians on the abdomen are determined based on the human body key feature points.

In step S103, abdominal acupuncture points are determined based on a topological relationship of the meridians and acupuncture points.

The human body image in step S101 may be acquired in the following manners: the human body image is shot by an image acquisition device (e.g., a camera) in real time; or the human body image is fetched from a storage medium storing pre-shot human body images.

Compared with the prior art, the method for determining abdominal acupuncture points provided in the embodiments of the present disclosure can determine a plurality of meridians on an abdomen based on human body key feature points and then determine abdominal acupuncture points based on a topological relationship of the meridians and acupuncture points. In this way, it is realized that the abdominal acupuncture points are determined by means of the meridians such that the technical problem of inaccurate locating of acupuncture points on the abdomen without texture feature due to the use of a deep learning model is avoided, and the locating accuracy of the abdominal acupuncture points is improved.

Further, the embodiments of the present disclosure omit a marking process without marking acupuncture points. Therefore, the problem of inaccurate locating caused by inaccurate marking can be avoided. The locating accuracy of the abdominal acupuncture points is further improved, and the locating efficiency of the abdominal acupuncture points is improved.

In order to improve the efficiency and accuracy of acquiring the human body key feature points, in some embodiments of the present disclosure, the human body key feature points include a first-type human body key feature point and a second-type human body key feature point; and as shown in FIG. 2, step S101 includes the following steps.

In step S201, a key point is extracted from the human body image based on a Mediapipe extraction model to determine the first-type human body key feature point.

In step S202, an initial detection model is trained to obtain a target detection model, and a key point is extracted from the human body image based on the target detection model to determine the second-type human body key feature point.

This is because an open-source Mediapipe extraction model can extract 33 skeleton key points of the human body, including the first-type human body key feature point. Therefore, the first-type human body key feature point is determined by the open-source Mediapipe extraction model that is used directly without training so that the efficiency of determining the first-type human body key feature point can be improved. Further, the Mediapipe extraction model is trained and verified with a large quantity of sample data and has high accuracy. Therefore, the accuracy of the determined first-type human body key feature point can be improved.

For the second-type human body key feature point that cannot be extracted by the Mediapipe extraction model, the target detection model is established and trained, and the second-type human body key feature point is extracted based on the target detection model, and the accuracy of the second-type human body key feature point is improved.

The target detection model may be any deep learning model, such as Faster-CNN, SSD, and YOLO. In a specific embodiment of the present disclosure, the target detection model is YOLOv8.

A specific training process of the initial detection model is as follows: 500 pictures are collected and enhanced to 1000 pictures by data enhancement. The navels and left and right nipples in the 1000 pictures are marked to obtain a training set. The initial detection model is trained based on the training set to obtain the target detection model. The mean average precision (mAP), accuracy, and recall rate of the target detection model meet requirements. Specifically, mAP@0.5 and mAP@0.5:0.95 reach 99.2% and 92.9%, respectively.

In some embodiments of the present disclosure, the first-type human body key feature point includes a nose; the second-type human body key feature point includes a left nipple, a right nipple, and a navel; and the meridians include a Conception Vessel, a left kidney meridian of foot-shaoyin, a right kidney meridian of foot-shaoyin, a left stomach meridian of foot-yangming, a right stomach meridian of foot-yangming, a left spleen meridian of foot-taiyin, and a right spleen meridian of foot-taiyin.

The left kidney meridian of foot-shaoyin and the right kidney meridian of foot-shaoyin, the left stomach meridian of foot-yangming and the right stomach meridian of foot-yangming, and the left spleen meridian of foot-taiyin and the right spleen meridian of foot-taiyin are bilaterally symmetrical about the Conception Vessel.

In other words, the first-type human body key feature point is a point that can be acquired by the open-source Mediapipe extraction model, and the second-type human body key feature point is a point having an obvious abdominal feature.

In the embodiments of the present disclosure, the second-type human body key feature point includes the left nipple, the right nipple, and the navel. Since the left nipple, the right nipple, and the navel have obvious features, the identification accuracy of the second-type human body key feature point can be improved, and the locating accuracy of the abdominal acupuncture points can be guaranteed.

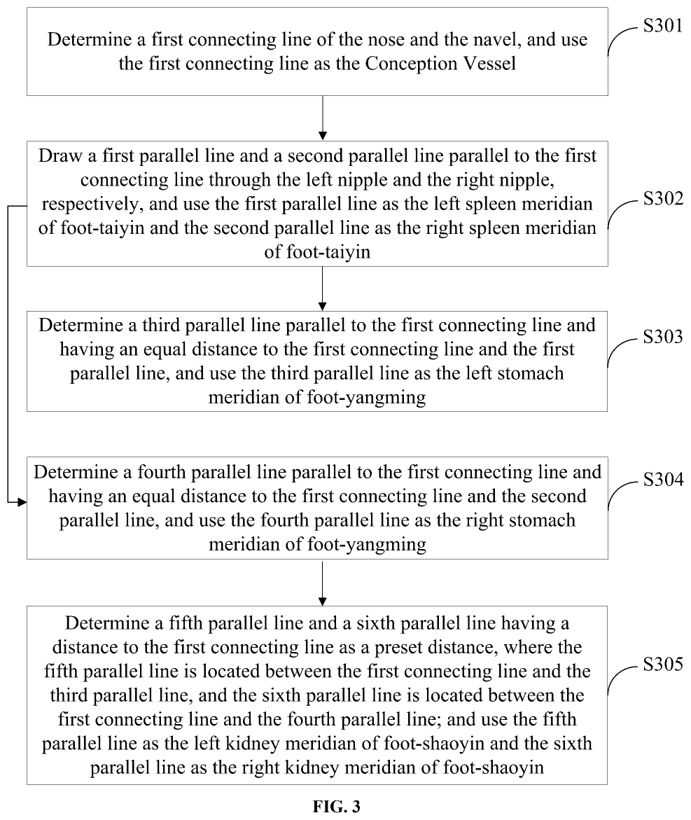

In some embodiments of the present disclosure, as shown in FIG. 3, step S102 includes following steps.

In step S301, a first connecting line of the nose and the navel is determined, and the first connecting line is used as the Conception Vessel L1.

In step S302, a first parallel line and a second parallel line parallel to the first connecting line through the left nipple and the right nipple are drawn, respectively, and the first parallel line is used as the left spleen meridian of foot-taiyin L2 and the second parallel line as the right spleen meridian of foot-taiyin L3.

In step S303, a third parallel line parallel to the first connecting line and having an equal distance to the first connecting line and the first parallel line is determined, and the third parallel line is used as the left stomach meridian of foot-yangming L4.

In step S304, a fourth parallel line parallel to the first connecting line and having an equal distance to the first connecting line and the second parallel line is determined, and the fourth parallel line is used as the right stomach meridian of foot-yangming L5.

In step S305, a fifth parallel line and a sixth parallel line having a distance to the first connecting line as a preset distance are determined, where the fifth parallel line is located between the first connecting line and the third parallel line, and the sixth parallel line is located between the first connecting line and the fourth parallel line; and the fifth parallel line is used as the left kidney meridian of foot-shaoyin L6 and the sixth parallel line as the right kidney meridian of foot-shaoyin L7.

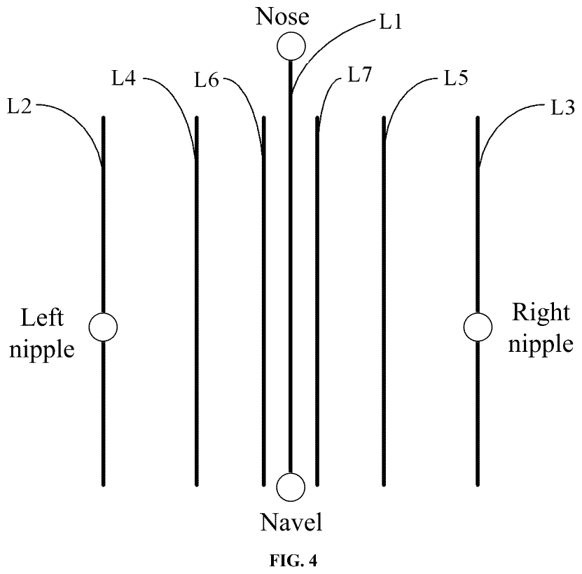

In a specific embodiment of the present disclosure, the Conception Vessel, the left kidney meridian of foot-shaoyin, the right kidney meridian of foot-shaoyin, the left stomach meridian of foot-yangming, the right stomach meridian of foot-yangming, the left spleen meridian of foot-taiyin, and the right spleen meridian of foot-taiyin are as shown in FIG. 4.

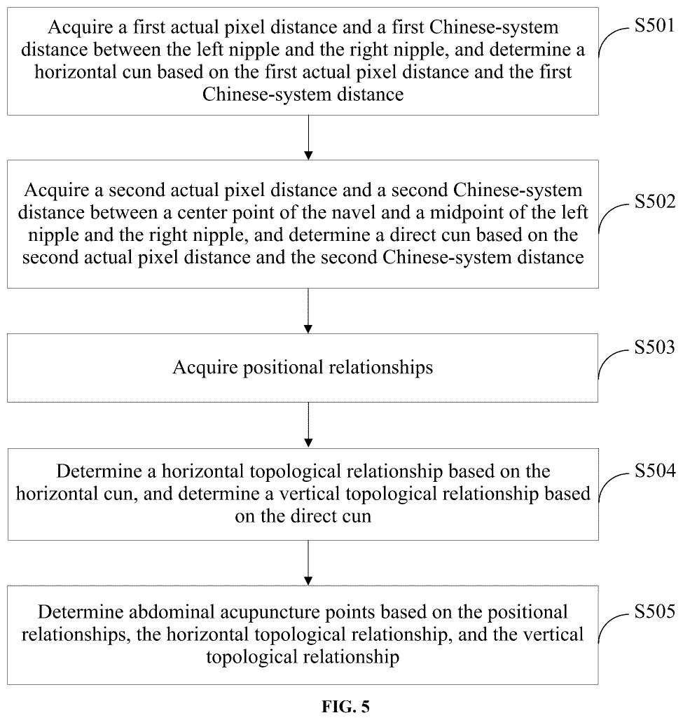

In some embodiments of the present disclosure, the topological relationship includes positional relationships between the meridians and the acupuncture points, a horizontal topological relationship between the meridians, and a vertical topological relationship between the acupuncture points; and as shown in FIG. 5, step S103 includes the following steps.

In step S501, a first actual pixel distance and a first Chinese-system distance between the left nipple and the right nipple are acquired, and a horizontal cun is determined based on the first actual pixel distance and the first Chinese-system distance.

In step S502, a second actual pixel distance and a second Chinese-system distance between a center point of the navel and a midpoint of the left nipple and the right nipple are acquired, and a direct cun is determined based on the second actual pixel distance and the second Chinese-system distance.

In step S503, the positional relationships are acquired.

In step S504, the horizontal topological relationship is determined based on the horizontal cun, and the vertical topological relationship based on the direct cun is determined.

In step S505, the abdominal acupuncture points are determined based on the positional relationships, the horizontal topological relationship, and the vertical topological relationship.

The Chinese-system distance is represented by cun, and the preset distance in step S305 is 0.5 horizontal cun.

In a specific embodiment of the present disclosure, the horizontal cun w is as follows:

w=b/l₁

The direct cun h is as follows:

h=d/l₂
where b represents the first actual pixel distance; 11 represents the first Chinese-system distance; d represents the second actual pixel distance; and 12 represents the second Chinese-system distance.

In a specific embodiment of the present disclosure, the first Chinese-system distance is 8 cun, and the second Chinese-system distance is 11 cun.

In a specific embodiment of the present disclosure, the abdominal acupuncture points include Shenque, Xiawan, Zhongwan, Shangwan, Qihai, Guanyuan, Zhongji, left Chengman, left Liangmen, left Taiyi, left Tianshu, left Shuidao, left Guilai, right Chengman, right Liangmen, right Taiyi, right Tianshu, right Shuidao, right Guilai, left Daheng, left Fujie, right Daheng, right Fujie, left Futonggu, left Yindu, left Shangqu, left huangshu, left Qixue, left Dahe, right Futonggu, right Yindu, right Shangqu, right huangshu, right Qixue, and right Dahe.

The positional relationships are as follows: Shenque, Xiawan, Zhongwan, Shangwan, Qihai, Guanyuan, and Zhongji are located on the Conception Vessel; left Chengman, left Liangmen, left Taiyi, left Tianshu, left Shuidao, and left Guilai are located on the left stomach meridian of foot-yangming; right Chengman, right Liangmen, right Taiyi, right Tianshu, right Shuidao, and right Guilai are located on the right stomach meridian of foot-yangming; left Daheng and left Fujie are located on the left spleen meridian of foot-taiyin; right Daheng and right Fujie are located on the right spleen meridian of foot-taiyin; left Futonggu, left Yindu, left Shangqu, left huangshu, left Qixue, and left Dahe are located on the left kidney meridian of foot-shaoyin; and right Futonggu, right Yindu, right Shangqu, right huangshu, right Qixue, and right Dahe are located on the right kidney meridian of foot-shaoyin.

The horizontal topological relationship is as follows: distances of the Conception Vessel to the left stomach meridian of foot-yangming and the right stomach meridian of foot-yangming are 2 horizontal cun; distances of the Conception Vessel to the left spleen meridian of foot-taiyin and the right spleen meridian of foot-taiyin are 4 horizontal cun; and distances of the Conception Vessel to the left kidney meridian of foot-shaoyin and the right kidney meridian of foot-shaoyin are 0.5 horizontal cun.

The vertical topological relationship is as follows: Xiawan, Zhongwan, and Shangwan are located between Shenque and the nose; a distance between Xiawan and Shenque is 2 direct cun; a distance between Zhongwan and Shenque is 4 direct cun; a distance between Shangwan and Shenque is 5 direct cun; Qihai, Guanyuan, and Zhongji are located between Shenque and a foot; a distance between Qihai and Shenque is 1.5 direct cun; a distance between Guanyuan and Shenque is 3 direct cun; a distance between Zhongji and Shenque is 4 direct cun; left Chengman, right Chengman, and Shangwan have a same vertical coordinate; left Liangmen, right Liangmen, left Yindu, right Yindu, and Zhongwan have a same vertical coordinate; left Taiyi, right Taiyi, left Shangqu, right Shangqu, and Xiawan have a same vertical coordinate; left Tianshu, right Tianshu, left Daheng, right Daheng, left Huangshu, right Huangshu, and Shenque have a same vertical coordinate; left Fujie, right Fujie, and Qihai have a same vertical coordinate; left Shuidao, right Shuidao, left Qixue, right Qixue, and Guanyuan have a same vertical coordinate; and left Guilai, right Guilai, left Dahe, right Dahe, and Zhongji have a same vertical coordinate.

If the coordinates of the center of the navel are [xo, yo]. Any point is used as the origin of coordinates; the direction from left to right is the forward direction of the X-axis, and the direction from head to feet is the forward direction of the Y-axis. The topological relationship of the abdominal acupuncture points and the coordinates are as shown in Table 1.

TABLE 1
Abdominal Acupuncture Points

| Name of | | |
|---|---|---|
| Acupuncture | Position of | Coordinates of |
| Point | Acupuncture Point | Acupuncture Point |
| Shenque | on the Conception Vessel | [x₀, y₀] |
| | through the center of the | |
| | navel | |
| Xiawan | on the Conception Vessel 2 | [x₀, y₀ − 2 h] |
| | cun above Shenque | |
| Zhongwan | on the Conception Vessel 4 | [x₀, y₀ − 4 h] |
| | cun above Shenque | |
| Shangwan | on the Conception Vessel 5 | [x₀, y₀ − 5 h] |
| | cun above Shenque | |
| Chengman | on the stomach meridian of | left [x₀ − 2 w, y₀ − 5 h] |
| (bilaterally | foot-yangming 2 cun beside | right [x₀ + 2 w, y₀ − 5 h] |
| symmetrical) | Shangwan | |
| Liangmen | on the stomach meridian of | left [x₀ − 2 w, y₀ − 4 h] |
| (bilaterally | foot-yangming 2 cun beside | right [x₀ + 2 w, y₀ − 4 h] |
| symmetrical) | Zhongwan | |
| Taiyi | on the stomach meridian of | left [x₀ − 2 w, y₀ − 2 h] |
| (bilaterally | foot-yangming 2 cun beside | right [x₀ + 2 w, y₀ − 2 h] |
| symmetrical) | Xiawan | |
| Tianshu | on the stomach meridian of | left [x₀ − 2 w, y₀] |
| (bilaterally | foot-yangming 2 cun beside | right [x₀ + 2 w, y₀] |
| symmetrical) | Shenque | |
| Daheng | on the spleen meridian of | left [x₀ − 4 w, y₀] |
| (bilaterally | foot-taiyin 4 cun beside | right [x₀ + 4 w, y₀] |
| symmetrical) | Shenque | |
| Qihai | on the Conception Vessel | [x₀, y₀ + 1.5 h] |
| | 1.5 cun below Shenque | |

Specifically, in the direction from head to feet, the abdominal acupuncture points on the Conception Vessel are sequentially as follows: Shangwan, Zhongwan, Xiawan, Shenque, Qihai, Guanyuan, and Zhongji; the abdominal acupuncture points on the spleen meridian of foot-taiyin are sequentially as follows: Daheng and Fujie; the abdominal acupuncture points on the stomach meridian of foot-yangming are sequentially as follows: Chengman, Liangmen, Taiyi, Tianshu, Shuidao, and Guilai; and the abdominal acupuncture points on the kidney meridian of foot-shaoyin are sequentially as follows: Futonggu, Yindu, Shangqu, Huangshu, Qixue, and Dahe.

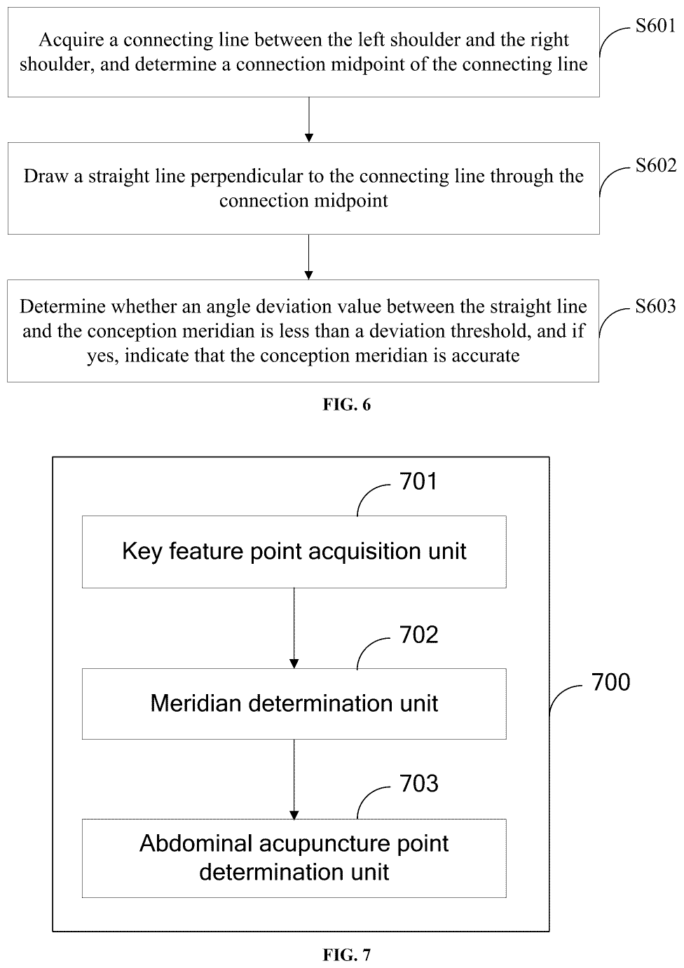

From the descriptions of the above embodiment, it can be known that all other meridians are determined on the basis of the Conception Vessel. Therefore, the accurate determination of the Conception Vessel is crucial for the locating accuracy of the abdominal acupuncture points. In order to ensure the accuracy of the Conception Vessel, in some embodiments of the present disclosure, the first-type human body key feature point further includes a left shoulder and a right shoulder; and as shown in FIG. 6, prior to step S103, the method further includes the following steps.

In step S601, a connecting line between the left shoulder and the right shoulder is acquired, and a connection midpoint of the connecting line is determined.

In step S602, a straight line perpendicular to the connecting line through the connection midpoint is drawn.

In step S603, whether an angle deviation value between the straight line and Conception Vessel is less than a deviation threshold is determined, and if yes, the Conception Vessel is accurate.

The embodiments of the present disclosure propose other way of determining the Conception Vessel than the way of determining the Conception Vessel based on the nose and the navel, and the Conception Vessel determined by this way of determining the Conception Vessel is used to validate the Conception Vessel determined based on the nose and the navel. The accuracy of the determined Conception Vessel can be ensured, and the locating accuracy of the abdominal acupuncture points is improved.

It should be understood that the deviation threshold may be set or adjusted according to an actual application scenario or requirement, which will not be particularly limited here.

It needs to be noted that an actual process of applying moxibustion by the method for locating abdominal acupuncture points is as follows: firstly, an abdominal acupuncture point is determined based on the method for locating abdominal acupuncture points, and in this case, the abdominal acupuncture point is coordinate values in a camera coordinate system. The abdominal acupuncture point is then converted to coordinates in a world coordinate system based on a conversion relationship between the camera coordinate system and the world coordinate system. The coordinates are transmitted to a moxibustion applying device, and the moxibustion applying device can automatically apply moxibustion based on the coordinates.

In order to better implement the method for determining abdominal acupuncture points in the embodiments of the present disclosure, on the basis of the method for determining abdominal acupuncture points, correspondingly, an embodiment of the present disclosure further provides an apparatus for determining abdominal acupuncture points. As shown in FIG. 7, the apparatus 700 for determining abdominal acupuncture points includes:
a key feature point acquisition unit 701 configured to acquire human body key feature points of a human body image;
a meridian determination unit 702 configured to determine a plurality of meridians on an abdomen based on the human body key feature points; and
an abdominal acupuncture point determination unit 703 configured to determine abdominal acupuncture points based topological relationship of the meridians and acupuncture points.

The apparatus 700 for determining abdominal acupuncture points provided in the above embodiment can implement the technical solutions described in the above embodiments of the method for determining abdominal acupuncture points. The specific implementation principles of the above modules or units may be known with reference to the corresponding contents in the above embodiments of the method for determining abdominal acupuncture points, which will not be redundantly described here.

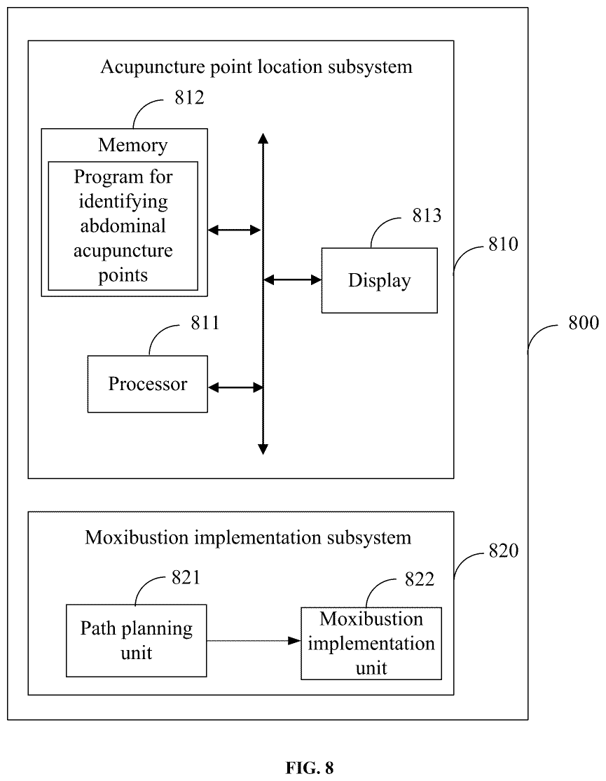

As shown in FIG. 8, correspondingly, the present disclosure further provides a moxibustion robot 800. The moxibustion robot 800 includes an acupuncture point location subsystem 810 for determining abdominal acupuncture points and a moxibustion implementation subsystem 820 for applying moxibustion based on the abdominal acupuncture points. The acupuncture point location subsystem 810 includes a processor 811, a memory 812, and a display 813. FIG. 8 only shows part of components of the moxibustion robot 800. However, it should be understood that not all the shown components need to be implemented, and more or less components may be implemented instead.

In some embodiments, the memory 812 may be an internal storage unit of the acupuncture point location subsystem 810, for example, a hard disk or an internal storage of the acupuncture point location subsystem 810. In some other embodiments, the memory 812 may also be an external storage device of the acupuncture point location subsystem 810, such as a plug-in hard disk, a smart media card (SMC), a secure digital (SD) card, or a flash card that is equipped on the acupuncture point location subsystem 810.

Further, the memory 812 may include both an internal storage unit and an external storage device of the acupuncture point location subsystem 810. The memory 812 is configured to store and install application software and various types of data of the acupuncture point location subsystem 810.

In some embodiments, the processor 811 may be a central processing unit (CPU), a microprocessor, or other data processing chips, and is configured to run a program code stored in the memory 812 or process data, e.g., the method for determining abdominal acupuncture points in the present disclosure.

In some embodiments, the display 813 may be a light-emitting diode (LED) display, a liquid crystal display, a touch liquid crystal display, an organic light-emitting diode (OLED) touch device, etc. The display 813 is configured to display the information of the acupuncture point location subsystem 810 and to display a visual user interface. Components 811-813 of the acupuncture point location subsystem 810 communicate with one another through a system bus.

In some embodiments of the present disclosure, when the processor 811 executes a program for identifying abdominal acupuncture points in the memory 812, the following steps may be implemented.

Human body key feature points of a human body image are acquired.

A plurality of meridians on the abdomen are determined based on the human body key feature points.

Abdominal acupuncture points are determined based on a topological relationship of the meridians and acupuncture points.

It should be understood that when the processor 811 executes the program for identifying abdominal acupuncture points in the memory 812, in addition to the above functions, other functions may also be implemented, as specifically described in the foregoing related method embodiments.

As shown in FIG. 8, the moxibustion implementation subsystem 820 includes a path planning unit 821 and a moxibustion implementation unit 822. The path planning unit 821 is configured to plan a path according to a position of an acupuncture needle in the moxibustion implementation unit 822 and coordinates of the acupuncture points to generate a planned path. The moxibustion implementation unit 822 is configured to move the acupuncture needle to the positions of the acupuncture points according to the planned path.

The moxibustion robot 800 provided in the embodiments of the present disclosure can perform the moxibustion treatment for a long time instead of a doctor. The burden of the doctor can be reduced and the medical consumption can be reduced.

Correspondingly, an embodiment of the present disclosure further provides a computer readable storage medium configured to store computer readable programs or instructions which, when executed, can implement the steps or functions of the method for determining abdominal acupuncture points provided in the above method embodiments.

Those skilled in the art can understand that relevant hardware (such as a processor and a controller) can be instructed by computer programs to implement all or part of processes of the method of the above embodiments, and the computer programs can be stored in the computer-readable storage medium. The computer readable storage medium is a magnetic disk, an optical disk, a read-only memory (ROM), a random access memory (RAM), or the like.

The method and apparatus for determining abdominal acupuncture points, the moxibustion robot, and the storage medium provided in the present disclosure are described above in detail. Several examples are used herein for illustration of the principles and implementations of the present disclosure. The description of the above embodiments is used to help understand the method of the present disclosure and its core ideas. Meanwhile, those skilled in the art can make changes to the specific implementations and the application scope according to the ideas of the present disclosure. In conclusion, the contents of the specification shall not be construed as limitations to the present disclosure.

1. A method for determining abdominal acupuncture points, comprising:
acquiring human body key feature points of a human body image, wherein the human body key feature points comprise a first-type human body key feature point and a second-type human body key feature point; the first-type human body key feature point comprises a nose;
and the second-type human body key feature point comprises a left nipple, a right nipple, and a navel;
determining a plurality of meridians on an abdomen based on the human body key feature points, wherein the meridians comprise a Conception Vessel, a left kidney meridian of foot-shaoyin, a right kidney meridian of foot-shaoyin, a left stomach meridian of foot-yangming, a right stomach meridian of foot-yangming, a left spleen meridian of foot-taiyin, and a right spleen meridian of foot-taiyin; and
determining abdominal acupuncture points based on a topological relationship of the meridians and acupuncture points;
wherein the acquiring human body key feature points of a human body image comprises:
extracting a key point from the human body image based on a key point extraction model to determine the first-type human body key feature point; and
training an initial detection model to obtain a target detection model, and extracting a key point from the human body image based on the target detection model to determine the second-type human body key feature point; and
the determining a plurality of meridians on an abdomen based on the human body key feature points comprises:
determining a first connecting line of the nose and the navel, and using the first connecting line as the Conception Vessel;
drawing a first parallel line and a second parallel line parallel to the first connecting line through the left nipple and the right nipple, respectively, and using the first parallel line as the left spleen meridian of foot-taiyin and the second parallel line as the right spleen meridian of foot-taiyin;
determining a third parallel line parallel to the first connecting line and having an equal distance to the first connecting line and the first parallel line, and using the third parallel line as the left stomach meridian of foot-yangming;
determining a fourth parallel line parallel to the first connecting line and having an equal distance to the first connecting line and the second parallel line, and using the fourth parallel line as the right stomach meridian of foot-yangming; and
determining a fifth parallel line and a sixth parallel line having a distance to the first connecting line as a preset distance, wherein the fifth parallel line is located between the first connecting line and the third parallel line, and the sixth parallel line is located between the first connecting line and the fourth parallel line; and using the fifth parallel line as the left kidney meridian of foot-shaoyin and the sixth parallel line as the right kidney meridian of foot-shaoyin;
the topological relationship comprises positional relationships between the meridians and the acupuncture points, a horizontal topological relationship between the meridians, and a vertical topological relationship between the acupuncture points; and the determining the abdominal acupuncture points based on a topological relationship of the meridians and acupuncture points comprises:
acquiring a first actual pixel distance and a first Chinese-system distance between the left nipple and the right nipple, and determining a horizontal cun based on the first actual pixel distance and the first Chinese-system distance;
acquiring a second actual pixel distance and a second Chinese-system distance between a center point of the navel and a midpoint of the left nipple and the right nipple, and determining a direct cun based on the second actual pixel distance and the second Chinese-system distance;
acquiring the positional relationships;
determining the horizontal topological relationship based on the horizontal cun, and determining the vertical topological relationship based on the direct cun; and
determining the abdominal acupuncture points based on the positional relationships, the horizontal topological relationship, and the vertical topological relationship;
the abdominal acupuncture points comprise Shenque, Xiawan, Zhongwan, Shangwan, Qihai, Guanyuan, Zhongji, left Chengman, left Liangmen, left Taiyi, left Tianshu, left Shuidao, left Guilai, right Chengman, right Liangmen, right Taiyi, right Tianshu, right Shuidao, right Guilai, left Daheng, left Fujie, right Daheng, right Fujie, left Futonggu, left Yindu, left Shangqu, left huangshu, left Qixue, left Dahe, right Futonggu, right Yindu, right Shangqu, right huangshu, right Qixue, and right Dahe;
the positional relationships are as follows: Shenque, Xiawan, Zhongwan, Shangwan, Qihai, Guanyuan, and Zhongji are located on the Conception Vessel; left Chengman, left Liangmen, left Taiyi, left Tianshu, left Shuidao, and left Guilai are located on the left stomach meridian of foot-yangming; right Chengman, right Liangmen, right Taiyi, right Tianshu, right Shuidao, and right Guilai are located on the right stomach meridian of foot-yangming; left Daheng and left Fujie are located on the left spleen meridian of foot-taiyin; right Daheng and right Fujie are located on the right spleen meridian of foot-taiyin; left Futonggu, left Yindu, left Shangqu, left huangshu, left Qixue, and left Dahe are located on the left kidney meridian of foot-shaoyin; and right Futonggu, right Yindu, right Shangqu, right huangshu, right Qixue, and right Dahe are located on the right kidney meridian of foot-shaoyin;
the horizontal topological relationship is as follows: distances of the Conception Vessel to the left stomach meridian of foot-yangming and to the right stomach meridian of foot-yangming are 2 horizontal cun; distances of the Conception Vessel to the left spleen meridian of foot-taiyin and the right spleen meridian of foot-taiyin are 4 horizontal cun; and distances of the Conception Vessel to the left kidney meridian of foot-shaoyin and the right kidney meridian of foot-shaoyin are 0.5 horizontal cun; and
the vertical topological relationship is as follows: Xiawan, Zhongwan, and Shangwan are located between Shenque and the nose; a distance between Xiawan and Shenque is 2 direct cun; a distance between Zhongwan and Shenque is 4 direct cun; a distance between Shangwan and Shenque is 5 direct cun; Qihai, Guanyuan, and Zhongji are located between Shenque and a foot; a distance between Qihai and Shenque is 1.5 direct cun; a distance between Guanyuan and Shenque is 3 direct cun; a distance between Zhongji and Shenque is 4 direct cun; left Chengman, right Chengman, and Shangwan have a same vertical coordinate; left Liangmen, right Liangmen, left Yindu, right Yindu, and Zhongwan have a same vertical coordinate; left Taiyi, right Taiyi, left Shangqu, right Shangqu, and Xiawan have a same vertical coordinate; left Tianshu, right Tianshu, left Daheng, right Daheng, left Huangshu, right Huangshu, and Shenque have a same vertical coordinate; left Fujie, right Fujie, and Qihai have a same vertical coordinate; left Shuidao, right Shuidao, left Qixue, right Qixue, and Guanyuan have a same vertical coordinate; and left Guilai, right Guilai, left Dahe, right Dahe, and Zhongji have a same vertical coordinate.

2. The method for determining abdominal acupuncture points according to claim 1, wherein the first-type human body key feature point further comprises a left shoulder and a right shoulder; and before the determining abdominal acupuncture points based on a topological relationship of the meridians and acupuncture points, the method further comprises:
acquiring a connecting line between the left shoulder and the right shoulder, and determining a midpoint of the connecting line;
drawing a straight line perpendicular to the connecting line through the connection midpoint; and
determining whether an angle deviation value between the straight line and the Conception Vessel is less than a deviation threshold, and if yes, indicating that the Conception Vessel is accurate.

3. An apparatus for determining abdominal acupuncture points, comprising:
a key feature point acquisition unit configured to acquire human body key feature points of a human body image, wherein the human body key feature points comprise a first-type human body key feature point and a second-type human body key feature point; the first-type human body key feature point comprises a nose; and the second-type human body key feature point comprises a left nipple, a right nipple, and a navel; a meridian determination unit configured to determine a plurality of meridians on an abdomen based on the human body key feature points, wherein the meridians comprise a Conception Vessel, a left kidney meridian of foot-shaoyin, a right kidney meridian of foot-shaoyin, a left stomach meridian of foot-yangming, a right stomach meridian of foot-yangming, a left spleen meridian of foot-taiyin, and a right spleen meridian of foot-taiyin; and
an abdominal acupuncture point determination unit configured to determine abdominal acupuncture points based on a topological relationship of the meridians and acupuncture points;
wherein the acquiring human body key feature points of a human body image comprises:
extracting a key point from the human body image based on a key point extraction model to determine the first-type human body key feature point; and
training an initial detection model to obtain a target detection model, and extracting a key point from the human body image based on the target detection model to determine the second-type human body key feature point; and
the determining a plurality of meridians on an abdomen based on the human body key feature points comprises:
determining a first connecting line of the nose and the navel, and using the first connecting line as the Conception Vessel;
drawing a first parallel line and a second parallel line parallel to the first connecting line through the left nipple and the right nipple, respectively, and using the first parallel line as the left spleen meridian of foot-taiyin and the second parallel line as the right spleen meridian of foot-taiyin;
determining a third parallel line parallel to the first connecting line and having an equal distance to the first connecting line and the first parallel line, and using the third parallel line as the left stomach meridian of foot-yangming;
determining a fourth parallel line parallel to the first connecting line and having an equal distance to the first connecting line and the second parallel line, and using the fourth parallel line as the right stomach meridian of foot-yangming; and
determining a fifth parallel line and a sixth parallel line having a distance to the first connecting line as a preset distance, wherein the fifth parallel line is located between the first connecting line and the third parallel line, and the sixth parallel line is located between the first connecting line and the fourth parallel line; and using the fifth parallel line as the left kidney meridian of foot-shaoyin and the sixth parallel line as the right kidney meridian of foot-shaoyin;
the topological relationship comprises positional relationships between the meridians and the acupuncture points, a horizontal topological relationship between the meridians, and a vertical topological relationship between the acupuncture points; and the determining the abdominal acupuncture points based on a topological relationship of the meridians and acupuncture points comprises:
acquiring a first actual pixel distance and a first Chinese-system distance between the left nipple and the right nipple, and determining a horizontal cun based on the first actual pixel distance and the first Chinese-system distance;
acquiring a second actual pixel distance and a second Chinese-system distance between a center point of the navel and a midpoint of the left nipple and the right nipple, and determining a direct cun based on the second actual pixel distance and the second Chinese-system distance;
acquiring the positional relationships;
determining the horizontal topological relationship based on the horizontal cun, and determining the vertical topological relationship based on the direct cun; and
determining the abdominal acupuncture points based on the positional relationships, the horizontal topological relationship, and the vertical topological relationship;
the abdominal acupuncture points comprise Shenque, Xiawan, Zhongwan, Shangwan, Qihai, Guanyuan, Zhongji, left Chengman, left Liangmen, left Taiyi, left Tianshu, left Shuidao, left Guilai, right Chengman, right Liangmen, right Taiyi, right Tianshu, right Shuidao, right Guilai, left Daheng, left Fujie, right Daheng, right Fujie, left Futonggu, left Yindu, left Shangqu, left huangshu, left Qixue, left Dahe, right Futonggu, right Yindu, right Shangqu, right huangshu, right Qixue, and right Dahe;
the positional relationships are as follows: Shenque, Xiawan, Zhongwan, Shangwan, Qihai, Guanyuan, and Zhongji are located on the Conception Vessel; left Chengman, left Liangmen, left Taiyi, left Tianshu, left Shuidao, and left Guilai are located on the left stomach meridian of foot-yangming; right Chengman, right Liangmen, right Taiyi, right Tianshu, right Shuidao, and right Guilai are located on the right stomach meridian of foot-yangming; left Daheng and left Fujie are located on the left spleen meridian of foot-taiyin; right Daheng and right Fujie are located on the right spleen meridian of foot-taiyin; left Futonggu, left Yindu, left Shangqu, left huangshu, left Qixue, and left Dahe are located on the left kidney meridian of foot-shaoyin; and right Futonggu, right Yindu, right Shangqu, right huangshu, right Qixue, and right Dahe are located on the right kidney meridian of foot-shaoyin;
the horizontal topological relationship is as follows: distances of the Conception Vessel to the left stomach meridian of foot-yangming and the right stomach meridian of foot-yangming are 2 horizontal cun; distances of the Conception Vessel to the left spleen meridian of foot-taiyin and the right spleen meridian of foot-taiyin are 4 horizontal cun; and distances of the Conception Vessel to the left kidney meridian of foot-shaoyin and the right kidney meridian of foot-shaoyin are 0.5 horizontal cun; and
the vertical topological relationship is as follows: Xiawan, Zhongwan, and Shangwan are located between Shenque and the nose; a distance between Xiawan and Shenque is 2 direct cun; a distance between Zhongwan and Shenque is 4 direct cun; a distance between Shangwan and Shenque is 5 direct cun; Qihai, Guanyuan, and Zhongji are located between Shenque and a foot; a distance between Qihai and Shenque is 1.5 direct cun; a distance between Guanyuan and Shenque is 3 direct cun; a distance between Zhongji and Shenque is 4 direct cun; left Chengman, right Chengman, and Shangwan have a same vertical coordinate; left Liangmen, right Liangmen, left Yindu, right Yindu, and Zhongwan have a same vertical coordinate; left Taiyi, right Taiyi, left Shangqu, right Shangqu, and Xiawan have a same vertical coordinate; left Tianshu, right Tianshu, left Daheng, right Daheng, left Huangshu, right Huangshu, and Shenque have a same vertical coordinate; left Fujie, right Fujie, and Qihai have a same vertical coordinate; left Shuidao, right Shuidao, left Qixue, right Qixue, and Guanyuan have a same vertical coordinate; and left Guilai, right Guilai, left Dahe, right Dahe, and Zhongji have a same vertical coordinate.

4. A moxibustion robot, comprising an acupuncture point location subsystem for determining abdominal acupuncture points and a moxibustion implementation subsystem for applying moxibustion based on the abdominal acupuncture points, wherein the acupuncture point location subsystem comprises a memory and a processor;
the memory is configured to store a program; and
the processor is coupled with the memory and configured to execute the program stored in the memory to implement the steps of the method for determining abdominal acupuncture points according to claim 1.

5. A computer readable storage medium, storing a program or instructions which, when executed by a processor, cause(s) the steps of the method for determining abdominal acupuncture points according to claim 1 to be implemented.

6. A moxibustion robot, comprising an acupuncture point location subsystem for determining abdominal acupuncture points and a moxibustion implementation subsystem for applying moxibustion based on the abdominal acupuncture points, wherein the acupuncture point location subsystem comprises a memory and a processor;
the memory is configured to store a program; and
the processor is coupled with the memory and configured to execute the program stored in the memory to implement the steps of the method for determining abdominal acupuncture points according to claim 2.

7. A computer readable storage medium, storing a program or instructions which, when executed by a processor, cause(s) the steps of the method for determining abdominal acupuncture points according to claim 2 to be implemented.
